(19) United States
(12) Patent Application Publication
(10) Pub. No.: US 2010/0105916 A1
(43) Pub. Date: Apr. 29, 2010

(54) Processes for Preparing Donepezil

(75) Inventors: Raju Mahadev Kharatkar, Vadodara (IN); Sachin Vinayakrao Pande, Vadodara (IN); Nilesh Liladhar Bonde, Vadodara (IN); Godfree Pavalu Fernandes, Vadodara (IN); Sugata Chatterjee, Vadodara (IN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sterling Biotech Limited, Vadodara (IN)

(21) Appl. No.: 12/428,677

(22) Filed: Apr. 23, 2009

(30) Foreign Application Priority Data
Oct. 29, 2008 (IN) 2318/MUM/2008

(51) Int. Cl. C07D 211/06

(52) U.S. Cl. 546/206

(57) ABSTRACT

A process for preparing highly pure donepezil, particularly donepezil hydrochloride, is provided comprising hydrogenating compound 1-benzyl-4-[(5,6-dimethoxy-1-indanon)-2-ylidene]methyl-piperidine or its hydrochloride salt with a noble metal catalyst or a derivative thereof, in the presence of at least one additive, wherein the additive contains at least one heteroatom, e.g., a sulfur, phosphorous, or nitrogen heteroatom.

### CROSS-REFERENCES TO RELATED APPLICATIONS

This application claims the benefit of Indian Patent Application No. 2318/Mum/2008 filed Oct. 29, 2008, which is incorporated herein by reference in its entirety.

### FIELD OF THE INVENTION

The invention relates to an improved process for preparing donepezil and pharmaceutically acceptable salts thereof.

### BACKGROUND OF THE INVENTION

Donepezil hydrochloride (1-benzyl-4-[(5,6-dimethoxy-1-indanon)-2-yl]methylpiperidine hydrochloride) is an acetylcholinesterase inhibitor used as the active ingredient in ARICEPT®, which is marketed for the treatment of Alzheimer's disease. Donepezil may also be effective in the treatment, prevention, remission, amelioration of various senile dementias such as Alzheimer type senile dementia; cerebrovascular accidents associated with, for example, a cerebral accident (e.g., cerebral hemorrhage or cerebral infarction), cerebral arteriosclerosis, or an external head wound; and aprosexia, lalopathy, hypobulia, emotional changes, memory disturbance, hallucinatory-paranoid syndrome, and behavioral changes, which are associated with, for example, encephalitis or cerebral palsy.

Donepezil, chemically identified as 1-benzyl-4-[(5,6-dimethoxy-1-indanon)-2-yl]methylpiperidine, is shown below as formula (I):

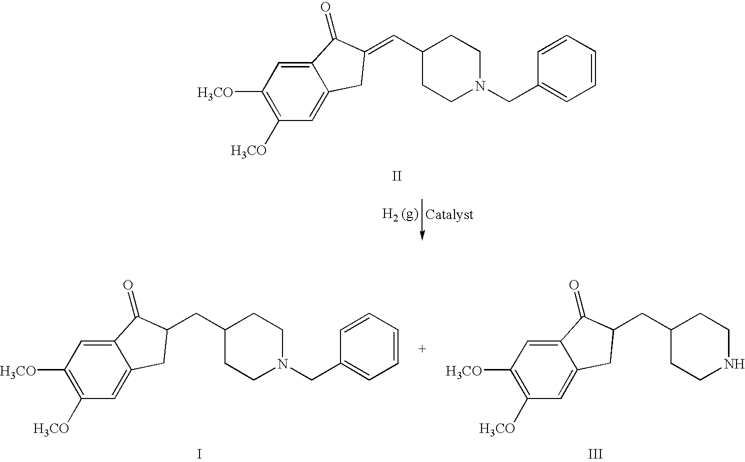

Donepezil (I) can be produced by catalytic hydrogenation of 1-benzyl-4-[(5,6-dimethoxy-1-indanon)-2-ylidene]methylpiperidine, the compound of formula (II). Because the starting compound has more than one functionality susceptible to hydrogenation, complete chemo-selectivity is unlikely. At least some N-debenzylation would likely occur resulting in the N-debenzylated impurity (III), 4-[(5,6-dimethoxy-1-indanon)-2-yl]methylpiperidine. Because catalytic hydrogenation is the last production step for the preparation of donepezil, there is a need for a hydrogenation step that minimizes or eliminates the N-debenzylated impurity.

U.S. Pat. No. 4,895,841 discloses preparing donepezil HCl by reacting 5,6-dimethoxy-1-indanone with 1-benzyl-4-formylpiperidine in the presence of a strong base such as lithium diisopropyl amide followed by reduction with hydrogen using palladium on carbon as catalyst (Examples 3 and 4). The overall yield of donepezil HCl was reported as 50.8%.

U.S. Pat. No. 5,606,064 discloses preparing donepezil HCl by reacting 5,6-dimethoxy-1-indanone with pyridine-4-aldehyde. The resulting 5,6-dimethoxy-2-(pyridin-4-yl)methylene-inda-1-one is reacted with benzyl bromide to afford 1-benzyl-4-[(5,6-dimethoxy-1-indanon-2-ylidene]methyl pyridinium bromide, which on reduction with hydrogen over platinum oxide catalyst afforded donepezil HCl (Examples 2, 4 and 6). The overall yield of donepezil HCl was reported as 58.5%.

WO 97/22584 discloses preparing donepezil HCl by oxalyl chloride chemistry. Pyridine-4-aldehyde was reacted with malonic acid. The resulting 3-(pyridin-4-yl)-2-propionic acid on reduction with rhodium on carbon under hydrogen atmosphere gave 3-(piperidin-4-yl)-2-propionic acid, which on reaction with methyl chlorocarbonate gave 3-[N-(methoxycarbonyl)piperidin-4-yl]propionic acid. On reacting 3-[N-(methoxycarbonyl)piperidin-4-yl]propionic acid with oxalyl chloride, methyl 4-(2-chlorocarbonylethyl)piperidin-1-carboxylate was obtained, which on reaction with 1,2-dimethoxy benzene in the presence of aluminum chloride afforded 4-[3-(3,4-dimethoxyphenyl)-3-oxopropyl]piperidin-1-carboxylate. On reacting 4-[3-(3,4-dimethoxyphenyl)-3-oxopropyl]piperidin-1-carboxylate with tetramethyl diamino methane, 4-[2-(3,4-dimethoxy benzoyl)allyl]piperidin-1-carboxylate was obtained, which on treatment with sulphuric acid gave methyl 4-(5,6-dimethoxy-1-indanon-2-yl methyl)piperidin-1-carboxylate. On decarboxylating 4-(5,6-dimethoxy-1-indanon-2-yl methyl)piperidin-1-carboxylate, 5,6-dimethoxy-2-(piperidin-4-yl methyl)-1-indanone was obtained, which on treatment with benzyl bromide afforded donepezil HCl (Example 1 to 6) in an overall yield reported as 19.3%.

U.S. Pat. No. 6,252,081 discloses preparing donepezil HCl by the reacting 1-indanone with a carbonate ester. The resulting 2-alkoxycarbonyl-1-indanone was halogenated with (4-pyridyl)methyl or a salt thereof and decarboxylated successively to give 2-(4-pyridyl)methyl-1-indanone derivative. On reacting the 2-(4-pyridyl)methyl-1-indanone derivative with benzyl bromide, their quaternary ammonium salts were formed, which on reduction with platinum oxide catalyst gave donepezil HCl (Examples 1 to 3). The overall yield of donepezil HCl was reported as 82%.

The prior art processes reduce compound (II) to donepezil (I). Reducing the double bond of compound (II) is frequently accompanied by the formation of side products, particularly N-debenzylated impurities, which are difficult to separate. The formation of side products, and the necessary purification steps to remove them, detrimentally affect product yield.

The alternative prior art processes using oxalyl chloride chemistry are not generally suitable for industrial scale production. The oxalyl reaction involves many time-consuming protection and de-protection steps, requires expensive raw materials such as chlorocarbonate or tetramethyl diaminomethane, and produces low overall yield.

Accordingly, there is a need for an industrially suitable process for preparing donepezil in a more efficient manner than heretofore possible.

### SUMMARY OF THE INVENTION

In one embodiment, a process for preparing donepezil, or an acid salt thereof, comprises hydrogenating the compound 1-benzyl-4-[(5,6-dimethoxy-1-indanon)-2-ylidene]methyl-piperidine or its hydrochloride salt with a noble metal catalyst or a derivative thereof, in the presence of at least one additive containing at least heteroatom, to form the product donepezil or an acid salt thereof substantially free of N-debenzylated impurities.

The catalyst can be, e.g., palladium, platinum, rhodium, or ruthenium or a derivative thereof. In one embodiment, the catalyst is palladium. The catalyst can be supported on a carrier, e.g., carbon, carbonate, metal sulfate, or alumina. In one embodiment, the carrier is carbon. The catalyst can be used in amount of about 1 to about 30, or about 10 to about 20 weight percent of the starting compound used, e.g., 1-benzyl-4-[(5,6-dimethoxy-1-indanon)-2-ylidene]methyl-piperidine.

In one embodiment, the additive contains at least one sulfur, nitrogen, or phosphorus heteroatom. In one embodiment, the additive is pyridine, thiourea, thioanisole, thiophenol, or triphenylphosphine. More than one additive may be used. The amount of the additive can be a small weight fraction of catalyst loading up to an equivalent weight of catalyst loading.

In one embodiment, the reaction takes place in the presence of at least one solvent such as, e.g., acetic acid, pyridine, methanol, ethanol, toluene, dichloromethane, tetrahydrofuran, and ethyl acetate. In one embodiment, the solvent is tetrahydrofuran.

In one embodiment, the reaction is conducted at about 0° C. to about 60° C., about 0° C. to about 30° C., or about 20° C. to about 30° C. In another embodiment, the reaction is conducted under hydrogen pressure of about 1 to about 100 bars, more preferably at about 1 bar.

In another embodiment, we provide donepezil or an acid salt thereof produced by hydrogenating 1-benzyl-4-[(5,6-dimethoxy-1-indanon)-2-ylidene]methylpiperidine in the presence of at least one additive containing at least one heteroatom, wherein the donepezil is at least 95% free of the N-debenzylated analog.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a reagent” is a reference to one or more reagents and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are detailed in the following description. It should be noted that the features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law.

In one embodiment, a process for preparing donepezil, or an acid salt thereof, comprises hydrogenating the compound 1-benzyl-4-[(5,6-dimethoxy-1-indanon)-2-ylidene]methylpiperidine (II) or its hydrochloride salt with a noble metal catalyst or a derivative thereof, in the presence of at least one additive, wherein the additive contains at least one heteroatom, to form the product donepezil (I) or an acid salt thereof. The process can include hydrogenating compound (II) to form donepezil (I), and then further converting donepezil (I) to an acid salt, e.g., hydrochloride salt, by conversions steps known in the art.

The processes described herein are also efficient, economical, and simple. In one embodiment, the process for preparing donepezil can be adapted for industrial scale up. In another embodiment, the process can advantageously give a high yield.

In one embodiment, the process advantageously yields highly pure product, that is, donepezil or its acid salt substantially free of impurities, particularly N-debenzylated impurities. Accordingly, in one embodiment the product is at least 75% free of N-debenzylated impurities, at least 80%, at least 90%, at least 95%, at least 97% at least 98%, at least 99%, at least 99.5%, or preferably at least 99.9% free of N-debenzylated impurities. In another embodiment, the product is at least 75% free of any impurities, preferably at least 80%, at least 90%, at least 95%, at least 97% at least 98%, at least 99%, or preferably at least 99.5% free of any impurities. In embodiments wherein the product is substantially free of any impurities, particularly N-debenzylated impurities, the need for subsequent purification steps, such as chromatography or recrystallization, can be reduced or eliminated.

In one embodiment, hydrogenation is performed using a palladium-carbon catalyst and at least one additive, at least one additive, wherein the additive contains at least one heteroatom, preferably a sulfur, phosphorous, or nitrogen heteroatom. This process is suitable for industrial production of donepezil (I) or its hydrochloride salt in high yield and without elaborate purification.

The noble metal catalyst can be palladium, platinum, rhodium, or ruthenium metal. Suitable derivatives of a noble metal catalyst include, but are not limited to, oxides, chlorides, or sulfates of the noble metal, e.g., palladium, platinum, rhodium, or ruthenium. In one embodiment, the noble metal catalyst is palladium.

The noble metal catalyst or derivative thereof can be supported on a carrier. Suitable materials for the carrier include, but are not limited to, carbon, alumina, carbonates, and metal sulfates. In one embodiment, the carrier includes carbon, calcium carbonate, barium sulfate, or alumina. In another embodiment, the carrier includes carbon.

In one embodiment, the additive contains at least one sulfur, phosphorous, or nitrogen heteroatom. In one embodiment, the additive contains at least one sulfur heteroatom. In another embodiment, the additive contains at least one phosphorus heteroatom. In yet another embodiment, the additive contains at least one nitrogen heteroatom. Exemplary additives include, but are not limited to, thiophenol, thioanisole, thiourea, triphenylphosphine, pyridine, or mixtures thereof. These additives are advantageously inexpensive, easily available, and safe.

The amount of additive used can be equivalent to or less than the weight of catalyst loading. In one embodiment, the amount of additive is a small weight fraction of catalyst loading. A small weight fraction can be, e.g., less than 0.5 eq, less than 0.2 eq, less than 0.1 eq, or less than 0.01 eq.

In one embodiment, at least one solvent is used for the catalytic hydrogenation. The solvent can be an acid, base, lower alkanol, or other organic solvent. Exemplary suitable solvents include, but are not limited to, dichloromethane, acetic acid, pyridine, methanol, ethanol, tetrahydrofuran, toluene, ethyl acetate, and mixtures thereof. Preferably, the solvent is tetrahydrofuran.

In another embodiment, the additive is a liquid phase at room temperature, and so the additive itself can be used as the solvent alone, or in combination with other solvents described above.

The reaction temperature for catalytic hydrogenation can be about 0° C. to about 60° C., about 0° C. to about 30° C., or about 20° C. to about 30° C.

Hydrogenation can comprise, e.g., blanketing the reaction mixture with hydrogen or bubbling hydrogen into the reaction mixture. Hydrogenation can be accomplished, e.g., under complete or partial hydrogenation atmosphere. The hydrogen pressure in the catalytic hydrogenation can be, e.g., about 1 to about 100 bars, about 1 to about 20 bars, or about 1 bar.

In another embodiment, donepezil or an acid salt thereof is provided, wherein the donepezil is produced by hydrogenation in the presence of at least one additive, wherein the additive contains at least one heteroatom. The donepezil is preferably substantially free of impurities, particularly N-debenzylated impurities, as described above.

Without further elaboration, it is believed that one skilled in the art using the preceding description can utilize the invention to the fullest extent. The following examples are illustrative only, and not limiting of the disclosure in any way whatsoever.

The disclosures of each reference and publication cited above is expressly incorporated by reference in its entirety to the same extent as if each were incorporated by reference individually.

### EXAMPLES

### Example 1

2 ml of a solution of 0.001% thioanisole in tetrahydrofuran and 0.4 g of 10% Pd on C were added to a solution of 2.0 g 1-benzyl-4-[(5,6-dimethoxy-1-indanon)-2-ylidene]methyl-piperidine in 50 ml tetrahydrofuran. The mixture was stirred for 5 to 10 minutes. Hydrogen was bubbled through the reaction mixture until the reaction was complete.

After completion of the reaction, the catalyst was filtered, and the filtrate was evaporated to yield donepezil in 90% yield with 98% chromatographic purity. Donepezil was subsequently converted to its HCl salt and purified to yield 1-benzyl-4-[5,6-dimethoxy-1-indanon)-2-yl]methyl piperidine hydrochloride with >99.5% chromatographic purity and <0.1% of the N-debenzylated impurity (III).

### Example 2

2 ml of a solution of 0.0005% thiourea in tetrahydrofuran and 0.4 g of 10% Pd on C were added to a solution of 2.0 g 1-benzyl-4-[(5,6-dimethoxy-1-indanon)-2-ylidene]methyl-piperidine in 50 ml tetrahydrofuran. The mixture was stirred for 5 to 10 minutes. Hydrogen was bubbled through the reaction mixture until the reaction was complete.

After completion of the reaction, the catalyst was filtered, and the filtrate was evaporated to yield donepezil in 90% yield with 98% chromatographic purity. Donepezil was subsequently converted to its HCl salt and purified to yield 1-benzyl-4-[5,6-dimethoxy-1-indanon)-2-yl]methyl piperidine hydrochloride with >99.5% chromatographic purity and <0.1% of the N-debenzylated impurity (III).

### Example 3

0.4 g of 10% Pd on C was added to a solution of 2.0 g 1-benzyl-4-[(5,6-dimethoxy-1-indanon)-2-ylidene]methyl-piperidine in 30 ml pyridine. The mixture was stirred for 5 to 10 minutes. Hydrogen was bubbled through the reaction mixture until the reaction was complete.

After completion of the reaction, the catalyst was filtered, and the filtrate was evaporated to yield donepezil in 90% yield with 98% chromatographic purity. Donepezil was subsequently converted to its HCl salt and purified to yield 1-benzyl-4-[5,6-dimethoxy-1-indanon)-2-yl]methyl piperidine hydrochloride with >99.5% chromatographic purity, and debenzylated impurity (III) was found to be below detection limit.

### Example 4

20 mg triphenylphosphine and 0.4 g of 10% Pd on C were added to a solution of 2.0 g 1-benzyl-4-[(5,6-dimethoxy-1-indanon)-2-ylidene]methyl-piperidine in 50 ml tetrahydrofuran. The mixture was stirred for 5 to 10 minutes. Hydrogen was then bubbled through the reaction mixture until the reaction was complete.

After completion of the reaction, the catalyst was filtered, the filtrate was evaporated, and the solid was washed with hexane to yield donepezil in 90% yield with 98% chromatographic purity. Donepezil was subsequently converted to its HCl salt and purified to yield 1-benzyl-4-[5,6-dimethoxy-1-indanon)-2-yl]methyl piperidine hydrochloride with >99.5% chromatographic purity and the N-debenzylated impurity (III) in <0.1% amount.

### Example 5

1 ml of a solution of 0.6% diphenyl sulphide in tetrahydrofuran and 0.4 g of 10% Pd on C were added to a solution of 2.0 g 1-benzyl-4-[(5,6-dimethoxy-1-indanon)-2-ylidene]methyl-piperidine in 50 ml tetrahydrofuran. The mixture was stirred for 5 to 10 minutes. Hydrogen was bubbled through the reaction mixture until the reaction was complete.

After completion of the reaction, the catalyst was filtered, and the filtrate was evaporated to yield donepezil in 90% yield with 98% chromatographic purity. The product was subsequently converted to its HCl salt and purified to yield 1-benzyl-4-[5,6-dimethoxy-1-indanon)-2-yl]methyl piperidine hydrochloride with >99.5% chromatographic purity and <0.1% of the N-debenzylated impurity (III).

### Example 6

5 ml of a solution of 0.1% sodium sulphide in methanol and 0.4 g of 10% Pd on C were added to a solution of 2.0 g 1-benzyl-4-[(5,6-dimethoxy-1-indanon)-2-ylidene]methyl-piperidine in 50 ml tetrahydrofuran. The mixture was stirred for 5 to 10 minutes. Hydrogen was bubbled through the reaction mixture until the reaction was complete.

After completion of the reaction, the catalyst was filtered, and the filtrate was evaporated to yield donepezil in 90% yield with 98% chromatographic purity. Donepezil was subsequently converted to its HCl salt and purified to yield 1-benzyl-4-[5,6-dimethoxy-1-indanon)-2-yl]methyl piperidine hydrochloride with >99.5% chromatographic purity and <0.1% of the N-debenzylated impurity (III).

### Comparative Example 7

All the above examples were carried out without using the additives and maintaining all the other conditions identical, resulting in the formation of N-debenzylated product (III) in amounts ranging from 20-26%.

What is claimed is:

1. A process for preparing donepezil, or an acid salt thereof, comprising: hydrogenating the compound 1-benzyl-4-[(5,6-dimethoxy-1-indanon)-2-ylidene]methyl-piperidine or its hydrochloride salt with a noble metal catalyst or a derivative thereof, in the presence of at least one additive containing at least one heteroatom, to form the product donepezil or an acid salt thereof, such that the donepezil or acid salt thereof is substantially free of N-debenzylated impurities.

2. The process of claim 1, wherein the catalyst is palladium, platinum, rhodium, or ruthenium or a derivative thereof.

3. The process of claim 2, wherein the catalyst is palladium.

4. The process of claim 1, wherein the catalyst is supported on a carrier comprising carbon, carbonate, metal sulfate, or alumina.

5. The process of claim 4, wherein the carrier comprises carbon.

6. The process of claim 5, wherein the catalyst is palladium on carbon.

7. The process of claim 1, wherein the catalyst is present in an amount of about 1 to about 30 weight percent of the 1-benzyl-4-[(5,6-dimethoxy-1-indanon)-2-ylidene]methyl-piperidine.

8. The process of claim 7, wherein the catalyst is present in an amount of about 10 to about 20 weight percent of the 1-benzyl-4-[(5,6-dimethoxy-1-indanon)-2-ylidene]methyl-piperidine.

9. The process of claim 1, wherein the additive contains at least one sulfur, nitrogen, or phosphorus heteroatom.

10. The process of claim 9, wherein the additive is pyridine, thiourea, thioanisole, thiophenol, or triphenylphosphine.

11. The process of claim 1, wherein the amount of the additive is a small weight fraction of catalyst loading up to an equivalent weight of catalyst loading.

12. The process of claim 1, wherein the additive is a liquid at room temperature.

13. The process of claim 1, wherein hydrogenating occurs in the presence of at least one solvent selected from the group consisting of acetic acid, pyridine, methanol, ethanol, toluene, dichloromethane, tetrahydrofuran, and ethyl acetate.

14. The process of claim 13, wherein the solvent is tetrahydrofuran.

15. The process of claim 1, wherein hydrogenating is performed at a temperature of about 0° C. to about 60° C.

16. The process of claim 15, wherein hydrogenating is performed at a temperature of about 0° C. to about 30° C.

17. The process of claim 16, wherein hydrogenating is performed at a temperature of about 20° C. to about 30° C.

18. The process of claim 1, wherein hydrogenating is performed under hydrogen atmosphere at a pressure of about 1 to about 100 bars.

19. The process of claim 18, wherein hydrogenating is performed under hydrogen atmosphere at a pressure of about 1 bar.

20. Donepezil or an acid salt thereof produced by hydrogenating 1-benzyl-4-[(5,6-dimethoxy-1-indanon)-2-ylidene]methylpiperidine in the presence of at least one additive containing at least one heteroatom, wherein the donepezil is at least 95% free of 4-[(5,6-dimethoxy-1-indanon)-2-yl]methylpiperidine.
